(19) United States
(12) Patent Application Publication
(10) Pub. No.: US 2025/0222234 A1
(43) Pub. Date: Jul. 10, 2025

(54) VASCULAR PUNCTURE SYSTEM AND METHOD FOR CONTROLLING THE SAME

(71) Applicant: TERUMO KABUSHIKI KAISHA, Tokyo (JP)

(72) Inventors: Takito INUKAI, Isehara-shi (JP); Yoichiro KUWANO, Princeton, NJ (US); Takumi FUKUDA, Hadano-shi (JP); Kazuki NAKASONE, Fujinomiya-shi (JP)

(73) Assignee: TERUMO KABUSHIKI KAISHA, Tokyo (JP)

(21) Appl. No.: 19/091,128

(22) Filed: Mar. 26, 2025

Related U.S. Application Data
(63) Continuation of application No. PCT/JP2023/034445, filed on Sep. 22, 2023.

(30) Foreign Application Priority Data
Sep. 29, 2022 (JP) 2022-156029

(51) Int. Cl. A61M 25/06; G16H 40/63

(52) U.S. Cl. CPC A61M 25/0612; G16H 40/63; A61M 2205/10; A61M 2205/18; A61M 2205/3327; A61M 2205/50; A61M 2210/04; A61M 2210/12

(57) ABSTRACT

A vascular puncture system capable of recognizing, using a cross-sectional image, a puncture state of a blood vessel at a desired position in a direction in which the blood vessel extends and a control method thereof are provided. The vascular puncture system punctures a blood vessel with a needle using an imaging unit which comes into contact with a skin surface and which obtains a cross-sectional image of a human body and a drive unit to which a needle having a sharp needle tip is connectable and which moves the needle in a puncture direction, the vascular puncture system including a movement unit that moves the imaging unit, and a control unit that receives information regarding a position of the needle and/or information regarding the cross-sectional image and that controls operation of the movement unit.

### CROSS-REFERENCES TO RELATED APPLICATIONS

This application is a continuation of International Application No. PCT/JP2023/034445 filed on Sep. 22, 2023, which claims priority to Japanese Application No. 2022-156029 filed on Sep. 29, 2022, the entire content of both of which is incorporated herein by reference.

### TECHNOLOGICAL FIELD

The present disclosure generally relates to a vascular puncture system capable of detecting a position of a blood vessel from an image obtained by an echograph and puncturing the blood vessel, and a method for controlling the vascular puncture system.

### BACKGROUND DISCUSSION

In order to secure an access route to a blood vessel for drug administration or endovascular treatment, vascular puncture is performed in which a human body is punctured with a needle having a sharp needle tip, the needle being covered by a flexible outer tube. The access route can be secured using the outer tube by inserting the outer tube into the blood vessel along with the needle and then removing only the needle. Since an operator cannot visually observe a blood vessel from a skin surface in the vascular puncture, the operator estimates a position of the blood vessel on the basis of standard knowledge of how blood vessels run and skills including tactile perception of vascular pulsation.

There are devices these years that identify a position of a blood vessel using a sensor and that automatically perform vascular puncture (see, for example, International Patent Application Publication No. WO 2019/235518 A).

In puncture of a radial artery by the way, for example, a method is performed in which a position of a blood vessel is identified on the basis of the operator's visual and tactile perception and a position of a needle tip with respect to the blood vessel is identified on the basis of presence or absence of backflow of blood from a needle. A method in which a needle is stuck into both a front wall and a rear wall of a blood vessel and pulled and removed from the rear wall, that is, so-called double-wall puncture (DWP), is widely used in order to increase a success rate of puncture and reduce the number of trials. Compared to a method for sticking a needle only into a front wall of a blood vessel, that is, so-called single-wall puncture (SWP), the DWP has no difference in terms of bleeding and occurrence of radial artery occlusion (RAO). Since the outer tube reaches a rear wall of a blood vessel after the blood vessel is punctured in the DWP, it is possible to suppress backflow of blood from the outer tube even when the needle is removed while leaving the outer tube.

In automatic puncture, an echogram is used to confirm a position of a puncture needle. When an echogram is obtained, an ultrasonic probe is brought into contact with the skin to capture a cross-sectional image of a blood vessel extending substantially parallel to the skin, so that the obtained echogram has a cross section substantially perpendicular to a direction in which the target blood vessel extends. Because the puncture needle is obliquely inserted into blood vessels with respect to directions in which the blood vessels extend, however, a needle tip can be imaged in the echogram only at one point until the needle tip reaches a rear wall of a blood vessel after passing through a front wall and the vicinity of a center of gravity of the blood vessel. For this reason, it is difficult to accurately grasp a positional relationship between a needle tip and vessel walls when puncture is performed using an apparatus.

### SUMMARY

A vascular puncture system is disclosed that is capable of recognizing, using a cross-sectional image, a puncture state of a blood vessel at a desired position in a direction in which the blood vessel extends and a method for controlling the vascular puncture system.

(1) A vascular puncture system is disclosed, which includes a vascular puncture system that punctures a blood vessel with a needle using an imaging unit which comes into contact with a skin surface and which obtains a cross-sectional image of a human body and a drive unit to which a needle having a sharp needle tip is connectable and which moves the needle in a puncture direction, the vascular puncture system including a movement unit that moves the imaging unit, and a control unit that receives information regarding a position of the needle and/or information regarding the cross-sectional image and that controls operation of the movement unit, in which the control unit calculates a puncture position on the blood vessel from the information regarding the position of the needle and/or the information regarding the cross-sectional image, and controls the operation of the movement unit in such a way as to move an imaging range of the imaging unit to the calculated puncture position.

Since the vascular puncture system according to (1) can change the imaging range of the imaging unit, it is possible to recognize, using the cross-sectional image, a puncture state of the blood vessel at a desired position in a direction in which the blood vessel extends.

(2) In the vascular puncture system according to (1), the control unit may be capable of obtaining positional information regarding a rear wall of the blood vessel to be punctured and moving the imaging range of the imaging unit from another position in such a way as to be able to image a puncture planned position of the rear wall. As a result, since the vascular puncture system can recognize whether or not the needle has reached the rear wall of the blood vessel and punctured the rear wall, it is possible to reliably puncture the rear wall with the needle and stop the needle at a desired position.

(3) In the vascular puncture system according to (1) or (2), the control unit may be capable of obtaining positional information regarding a center of gravity of the blood vessel to be punctured and moving the imaging range of the imaging unit from another position in such a way as to be able to image a position at or around the center of gravity through which the needle is planned to pass. As a result, since the vascular puncture system can recognize whether or not the needle has reached the center of gravity of the blood vessel, it is possible to reliably puncture the blood vessel with the needle such that the needle passes through the blood vessel and stop the needle at a desired position.

(4) In the vascular puncture system according to any one of (1) to (3), the control unit may be capable of obtaining positional information regarding a front wall of the blood vessel to be punctured and moving the imaging range of the imaging unit from another position in such a way as to be able to image a puncture planned position of the front wall. As a result, since the vascular puncture system can recognize whether or not the needle has reached the front wall of the blood vessel and punctured the front wall, it is possible to reliably puncture the front wall with the needle and stop the needle at a desired position.

(5) In the vascular puncture system according to any one of (1) to (4), the control unit may control the movement unit in such a way as to move the imaging range of the imaging unit before a puncture operation for controlling the drive unit in such a way as to move the needle in the puncture direction in order to puncture the blood vessel with the needle. As a result, since the vascular puncture system can set the imaging range in advance at a position to be imaged, it is possible to reliably grasp a state of the blood vessel while observing changes in the state of the blood vessel to be imaged.

(6) In the vascular puncture system according to any one of (1) to (5), the control unit may control the movement unit in such a way as to move the imaging range of the imaging unit during a puncture operation for controlling the drive unit in such a way as to move the needle in the puncture direction in order to puncture the blood vessel with the needle. As a result, the vascular puncture system can reduce time required for the movement of the imaging range and the puncture operation to reduce a burden on a patient. Furthermore, since the vascular puncture system can move the imaging range in accordance with the puncture operation, it is possible to continue to image a predetermined position of the needle, which moves in the puncture operation, and reliably grasp the state of the blood vessel in real-time.

(7) In the vascular puncture system according to any one of (1) to (6), the control unit may control the movement unit in such a way as to move the imaging range of the imaging unit after a puncture operation for controlling the drive unit in such a way as to move the needle in the puncture direction in order to puncture the blood vessel with the needle. As a result, since the vascular puncture system moves the imaging range after the puncture operation, it is possible to reliably grasp the state of the blood vessel to be imaged.

(8) In the vascular puncture system according to (6), the control unit may control the operation of the movement unit in accordance with a setting value for driving the drive unit. As a result, the vascular puncture system can move the imaging range in accordance with the movement of the needle driven by the drive unit.

(9) In the vascular puncture system according to (6), the control unit may control the operation of the movement unit such that the position of the needle identified from the cross-sectional image obtained from the imaging unit is maintained at a predetermined position. As a result, the vascular puncture system can move the imaging range in accordance with the movement of the needle driven by the drive unit.

(10) In the vascular puncture system according to any one of (1) to (9), the movement unit may move the imaging unit in such a way as to translate the cross- sectional image. As a result, the vascular puncture system can observe an entire imaging range under uniform conditions, it is possible to satisfactorily grasp a state of an observation target.

(11) In the vascular puncture system according to any one of (1) to (10), the movement unit may move the imaging unit in such a way as to tilt the cross-sectional image. As a result, the vascular puncture system need not slide the imaging unit with respect to the imaging target. When the imaging range is changed, therefore, it is possible to keep the imaging unit from being separated from the imaging target and becoming unable to obtain the image.

(12) In the vascular puncture system according to any one of (1) to (11), the movement unit may include a buffer that supports the imaging unit while absorbing displacement of the imaging unit in a direction toward an imaging target. As a result, when the imaging range is changed, the vascular puncture system can keep the imaging unit from being separated from the imaging target and becoming unable to obtain the image.

(13) In the vascular puncture system according to any one of (1) to (12), the control unit may cause, if the imaging by the imaging unit is abnormal, an information transmission unit that transmits information to an outside to transmit information indicating a warning. As a result, the vascular puncture system can issue a warning to the operator, and safety can be improved.

(14) A method for controlling a vascular puncture system is disclosed that includes an imaging unit which comes into contact with a skin surface and which obtains a cross-sectional image of a human body, a drive unit to which a needle having a sharp needle tip is connectable and which moves the needle in a puncture direction, a movement unit which moves the imaging unit, and a control unit which receives information regarding a position of the needle and/or information regarding the cross-sectional image and which controls operation of the drive unit and the movement unit, the method being performed by the control unit and including the steps of calculating a puncture position on the blood vessel from the information regarding the position of the needle and/or the information regarding the cross-sectional image, and controlling the operation of the movement unit in such a way as to move an imaging range of the imaging unit to the calculated puncture position.

Since the imaging range of the imaging unit is changed in the method of controlling a vascular puncture system according to (14), it is possible to recognize, using the cross-sectional image, a puncture state of the blood vessel at a desired position in a direction in which the blood vessel extends.

(15) A vascular puncture system is disclosed that includes a vascular puncture system that punctures a blood vessel with a needle having a sharp needle tip, the vascular puncture system including an imaging unit that comes into contact with a skin surface and that obtains a cross-sectional image of a human body, a drive unit to which the needle is connectable and that moves the needle to a puncture position on the blood vessel, a movement unit that moves the imaging unit, and a control unit that receives information regarding a position of the needle and/or information regarding the cross-sectional image and that controls operation of the drive unit and the movement unit, in which the control unit calculates the puncture position on the blood vessel from the information regarding the position of the needle and/or the information regarding the cross-sectional image, and controls the operation of the movement unit in such a way as to move an imaging range of the imaging unit to the calculated puncture position.

Since the imaging range of the imaging unit can be changed in the vascular puncture system according to (15), it is possible to recognize, using the cross-sectional image, a puncture state of the blood vessel at a desired position in a direction in which the blood vessel extends.

### BRIEF DESCRIPTION OF THE DRAWINGS

FIG. 1 is a side view of a vascular puncture system according to a first embodiment.

FIG. 2 is a top view of the vascular puncture system illustrating a positional relationship with an arm from which a cross-sectional image is obtained.

FIG. 3 is a configuration diagram of the vascular puncture system.

FIG. 4 is a schematic diagram illustrating an example of an image obtained by an imaging unit.

FIG. 5 is a side view illustrating the vascular puncture system immediately before puncture.

FIG. 6 is a top view illustrating the vascular puncture system immediately before the puncture.

FIGS. 7A and 7B are schematic diagrams for explaining a positional relationship between a blood vessel and a puncture unit in the first embodiment, where FIG. 7A illustrates a state in which a needle has passed through a center of gravity of the blood vessel, and FIG. 7B illustrates a state in which the needle has passed through a rear wall.

FIG. 8 is a flowchart illustrating a flow of control performed by a control unit according to the first embodiment.

FIGS. 9A and 9B are schematic diagrams for explaining a positional relationship between the blood vessel and the puncture unit in a second embodiment, where FIG. 9A illustrates a state in which the imaging unit is disposed at a position where a rear wall puncture planned position can be imaged, and FIG. 9B illustrates a state in which the needle has passed through a rear wall.

FIG. 10 is a flowchart illustrating a flow of control performed by the control unit according to the second embodiment.

FIG. 11 is a schematic view for explaining a positional relationship between the blood vessel and the puncture unit in a third embodiment, and illustrates a state in which the imaging unit is disposed at the position where the rear wall puncture planned position can be imaged after completion of the puncture.

FIG. 12 is a flowchart illustrating a flow of control performed by the control unit according to the third embodiment.

FIGS. 13A-13C are schematic views for explaining a positional relationship between the blood vessel and the puncture unit in a fourth embodiment, where FIG. 13A illustrates a state in which the imaging unit is disposed at a position where a front wall puncture planned position can be imaged, FIG. 13B illustrates a state in which the needle has passed through a center of gravity of the blood vessel, and FIG. 13C illustrates a state in which the needle has passed through the rear wall.

FIG. 14 is a flowchart illustrating a first half of a flow of control performed by the control unit according to the fourth embodiment.

FIG. 15 is a flowchart illustrating a second half of the flow of control performed by the control unit according to the fourth embodiment.

FIG. 16 is a flowchart illustrating a flow of control performed by the control unit according to a modification of the fourth embodiment.

FIG. 17 is a side view illustrating a modification of the vascular puncture system.

### DETAILED DESCRIPTION

Set forth below with reference to the accompanying drawings is a detailed description of embodiments of a vascular puncture system capable of detecting a position of a blood vessel from an image obtained by an echograph and puncturing the blood vessel, and a method for controlling the vascular puncture system. Note that dimensional ratios in the drawings might be exaggerated for convenience of description and differ from actual ratios.

### First Embodiment

A vascular puncture system 10 according to a first embodiment of the present invention is used to puncture an arm H of a human body, and obtains a cross-sectional image of the arm H, detects a position of an artery to be punctured, and automatically punctures the artery.

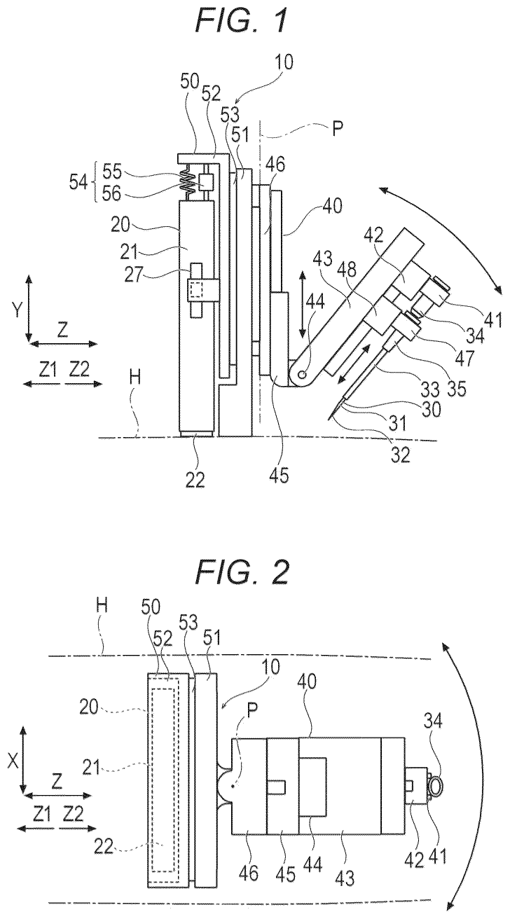

As illustrated in FIGS. 1 to 3, the vascular puncture system 10 includes an imaging unit 20 including a probe 22 that comes into contact with a skin surface and that obtains a cross-sectional image of a human body, a puncture unit 30 that performs puncture, a drive unit 40 that moves the puncture unit 30 with respect to the imaging unit 20, a movement unit 50 that moves an imaging range of the probe 22, a display unit 70 (information transmission unit) capable of displaying a cross-sectional image, and a control unit 60 that controls the imaging and the puncture.

The imaging unit 20 includes an imaging main body 21, the probe 22 disposed at a lower end of the imaging main body 21, a transmission portion 23 that transmits a signal from the control unit 60 to the probe 22, and a reception portion 24 that transmits a signal from the probe 22 to the control unit 60.

The probe 22 is provided at a central portion of a lower surface of the imaging unit 20 in such a way as to extend over substantially entire width. The probe 22 is an echograph including a transducer that generates an ultrasonic wave and that obtains a cross-sectional image of the inside of the human body by detecting the reflected ultrasonic wave. Since the cross-sectional image orthogonal to a direction in which a blood vessel extends is obtained in the present embodiment, the probe 22 is arranged such that a length direction of the probe 22 becomes orthogonal to a length direction of the arm H.

The probe 22 obtains a cross-sectional image as illustrated in FIG. 4. A horizontal direction in the cross-sectional image, that is, a width direction of the arm H, is defined as an X direction, a vertical direction in the cross-sectional image, that is, a depth direction of the arm H, is defined as a Y direction, and a direction orthogonal to a surface of the cross-sectional image, that is, the length direction of the arm H, is defined as a Z direction.

As illustrated in FIG. 1, the imaging main body 21 includes a linear slider 27 supported in such a way as to be linearly movable with respect to the movement unit 50. The linear slider is supported with respect to the movement unit 50 in such a way as to move the imaging main body in the Y direction, that is, in a direction in which the probe 22 approaches or departs from the arm H to be contacted.

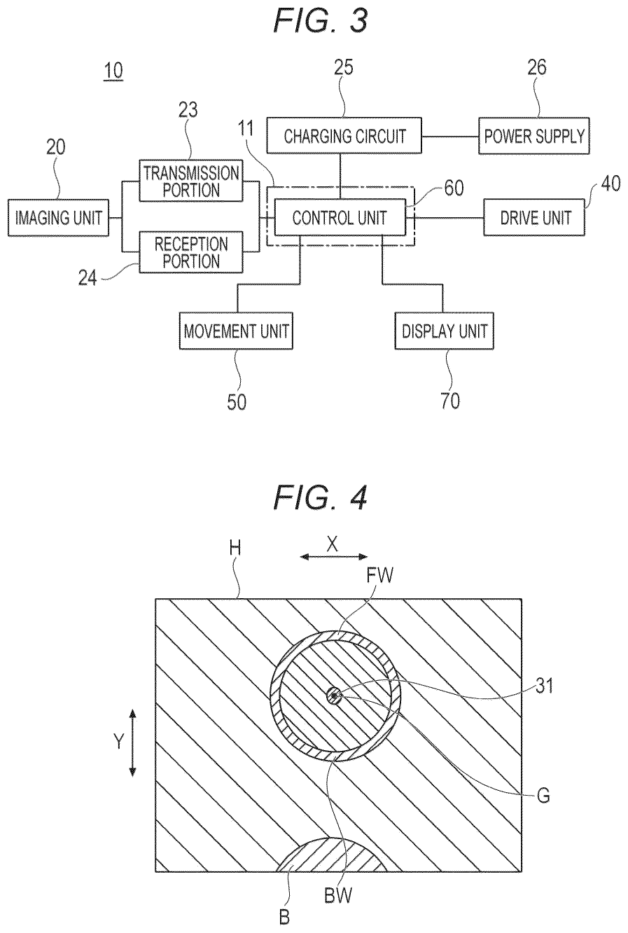

As illustrated in FIG. 3, the transmission portion 23 transmits a signal from the control unit 60 to the probe 22 in order to output an ultrasonic wave from the probe 22. The reception portion 24 transmits, to the control unit 60, a signal output from the probe 22 that has received a reflected wave.

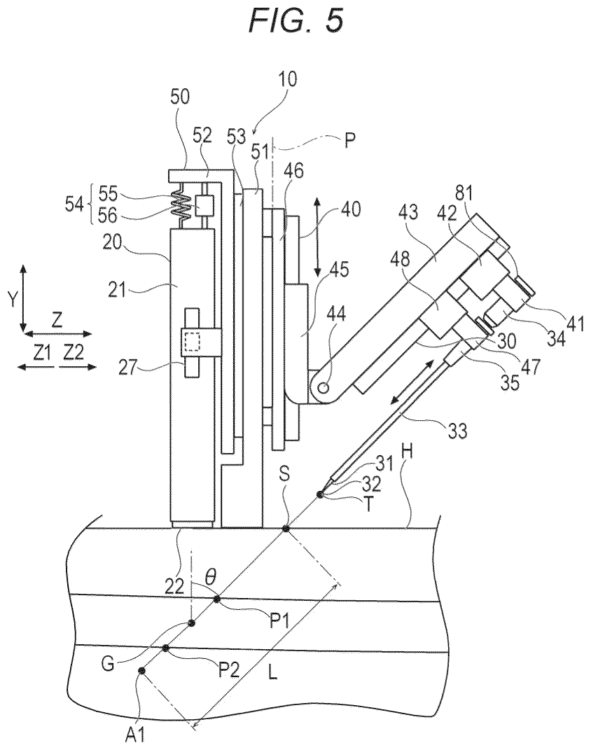

As illustrated in FIGS. 1 and 5, the puncture unit 30 includes a metal needle 31 with a sharp needle tip 32 at a distal end of the metal needle 31 and a flexible outer tube 33 disposed in such a way as to cover an outer peripheral surface of the needle 31. The needle 31 may be solid or hollow.

The needle tip 32 is a portion of the needle 31 having a blade surface inclined with respect to an axial center and located closer to the distal end than a portion where outer diameter is constant. Alternatively, the needle tip 32 may be a portion whose outer diameter decreases toward the most distal end that is sharp. The needle tip 32 protrudes from the outer tube 33 in a state where the outer tube 33 covers the needle 31. A needle hub 34 is fixed to a proximal end of the needle 31. A tubular outer tube hub 35 is fixed to a proximal end of the outer tube 33.

As illustrated in FIGS. 1 and 2, the drive unit 40 includes a first holding portion 41 that holds the needle hub 34, a first linear movement portion 42 that linearly moves the first holding portion 41, a second holding portion 47 that holds the outer tube hub 35, a second linear movement portion 48 that linearly moves the second holding portion 47, a tilting portion 43 that tilts the first holding portion 41 and the second holding portion 47, a third linear movement portion 45 that moves the tilting portion 43 in a length direction of the imaging unit 20, and a rotation portion 46 that rotates the third linear movement portion 45 about a predetermined rotation axis P.

The first holding portion 41 can detachably hold the needle hub 34. The first holding portion 41 can be, for example, a clamp that can sandwich and hold the needle hub 34.

The first linear movement portion 42 can linearly move the first holding portion 41 holding the needle hub 34 of the puncture unit 30 forward and backward in a direction in which the needle 31 extends (puncture direction). The first linear movement portion 42 is used to adjust a position of the needle 31 and puncture the blood vessel with the needle 31. The first linear movement portion 42 can include, for example, a rotary drive source, such as a motor, whose driving can be controlled by the control unit 60, and a structure (for example, a feed screw mechanism) that converts rotational motion of the rotary drive source into linear motion.

The second holding portion 47 can detachably hold the outer tube hub 35. The second holding portion 47 can be, for example, a clamp that can sandwich and hold the outer tube hub 35.

The second linear movement portion 48 can linearly move the second holding portion 47 holding the outer tube hub 35 of the puncture unit 30 forward and backward in a direction in which the outer tube 33 extends (puncture direction). The second linear movement portion 48 can adjust a position of the outer tube and push the outer tube 33 into a puncture hole formed by the needle 31. The second linear movement portion 48 can include, for example, a rotary drive source, such as a motor, whose driving can be controlled by the control unit 60, and a structure (for example, a feed screw mechanism) that converts rotational motion of the rotary drive source into linear motion.

The tilting portion 43 can tilt the first linear movement portion 42 and the second linear movement portion 48. The tilting portion 43 is used to change a puncture angle of the needle 31 and the outer tube 33 with respect to a skin surface of a patient. The tilting portion 43 includes a hinge 44 whose angle can be changed and a rotary drive source, such as a motor, whose driving can be controlled by the control unit 60 in order to change the angle of the hinge 44.

The third linear movement portion 45 is used to bring the puncture unit 30 close to or away from the patient's skin. The third linear movement portion 45 can linearly move the tilting portion 43 forward and backward in a direction in which the imaging unit 20 extends. The third linear movement portion 45 can include, for example, a rotary drive source, such as a motor, whose driving can be controlled by the control unit 60, and a structure (for example, a feed screw mechanism) that converts rotational motion of the rotary drive source into linear motion.

The rotation portion 46 is used to change a direction of the needle 31 when the third linear movement portion 45 is viewed substantially perpendicularly to the skin surface of the patient. The rotation portion 46 is capable of rotating the tilting portion 43 about the rotation axis P parallel to the length direction of the imaging unit 20. The rotation portion 46 can include, for example, a rotary drive source, such as a motor, whose driving can be controlled by the control unit 60.

The movement unit 50 includes a movement base 51 fixed to the rotation portion 46, a support 52 that supports the imaging unit 20, a movement mechanism 53 that moves the support with respect to the movement base, and a buffer 54 that supports the imaging unit 20 while absorbing an unintended displacement of the imaging unit 20.

The movement base 51 may be contactable with the arm H so as to be a base point of movement of the drive unit 40 and the movement unit 50. The support 52 is movable in the Z direction by the movement mechanism 53. The support 52 supports the imaging main body 21 via a linear slider.

The movement mechanism 53 can include, for example, a rotary drive source, such as a motor, whose driving can be controlled by the control unit 60, and a structure (for example, the feed screw mechanism) that converts rotational motion of the rotary drive source into linear motion. The movement mechanism 53 can move the imaging main body 21 in such a way as to translate the cross-sectional image obtained by the probe 22 in the Z direction, which is perpendicular to the cross-sectional image.

The buffer 54 includes a spring 55 and a damper 56 disposed between the support 52 and the imaging main body 21. The buffer 54 can press the probe 22 against a contact target using the spring 55 while absorbing, using the damper 56, kinetic energy of the imaging main body 21 applied to the support 52 in the Y direction, that is, in the direction in which the probe 22 approaches or departs from the arm H, which is the contact target. Note that the buffer 54 may include a drive source that actively moves the support 52 with respect to the movement base 51. As a result, during imaging, the control unit 60 can control the drive source of the buffer 54 such that the probe 22 of the imaging unit 20 is not separated from the arm H to be imaged.

The control unit 60 is preferably configured to be able to accurately control rotation and displacement of the drive sources used for the first linear movement portion 42, the second linear movement portion 48, the third linear movement portion 45, the rotation portion 46, and the movement unit 50 while grasping the rotation and displacement, and the drive sources are, for example, servomotors.

As illustrated in FIGS. 1 and 3, the control unit 60 transmits a signal to the probe 22 via the transmission portion 23 and causes the probe 22 to output an ultrasonic wave. In addition, the control unit 60 can form a cross-sectional image from a signal obtained from the probe 22 via the reception portion 24. Furthermore, the control unit 60 can cause the display unit 70 to display the obtained cross-sectional image. Furthermore, the control unit 60 can perform arithmetic processing such as an image analysis from information of the cross-sectional image to identify a position of a blood vessel in the image. Furthermore, the control unit 60 can control the operation of the drive unit 40 and the movement unit 50. The control unit 60 includes, as physical components, a storage circuit and an arithmetic circuit. The storage circuit can store programs and various parameters. The arithmetic circuit can perform arithmetic processing.

The control unit 60 is connected to a power supply 26 including a rechargeable battery via a charging circuit 25. The control unit 60 may be disposed in the imaging unit 20, the drive unit 40, or the movement unit 50, or may be configured separately from the imaging unit 20, the drive unit 40, or the movement unit 50.

The control unit 60 obtains a cross-sectional image as illustrated in FIG. 4 from the probe 22. Coordinates of an upper-left point in the cross-sectional image are defined as a start point (0, 0, 0). Note that in the cross-sectional image, a wall of a blood vessel on a side close to the skin to be punctured is a front wall FW, and a wall of the blood vessel on a side away from the skin to be punctured is a rear wall BW. A radius B might be located behind the rear wall BW. The puncture with the needle 31 is performed as double-wall puncture (DWP) so that the needle 31 passes through a front wall puncture planned position P1 of the front wall FW of the blood vessel, a center of gravity G, and a rear wall puncture planned position P2 of the rear wall BW. Since the needle 31 is inserted into blood vessels obliquely with respect to directions in which the blood vessels extend, however, the needle 31 penetrating the front wall FW and the needle 31 penetrating the rear wall BW cannot be observed in the cross-sectional image when a range including the center of gravity G of a blood vessel through which the needle 31 is to pass is observed in the cross-sectional image.

As illustrated in FIGS. 3 and 4, the display unit 70 (information notification unit) is a monitor or the like capable of displaying a cross-sectional image.

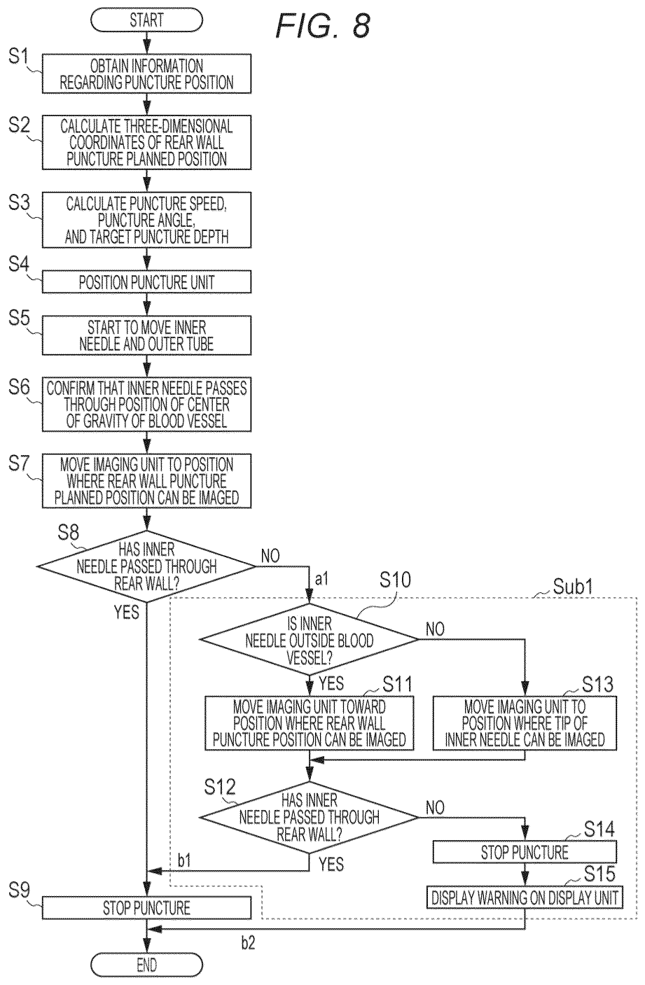

Next, a method for puncturing a blood vessel using the vascular puncture system 10 will be described with reference to a flowchart illustrated in FIG. 8 used by the control unit 60. As illustrated in FIGS. 1, 2 and 5, the vascular puncture system 10 is used in contact with the skin surface of the arm H.

The control unit 60 obtains information regarding a puncture position determined by another program (step S1). Calculation of the other program may be performed by the control unit 60 or may be performed by another device. When the puncture position is determined by another device, the control unit 60 is connected to the other device and obtains information from the other device. Information regarding positions of the needle 31 and the outer tube 33 is also input to the control unit 60. The information regarding the positions of the needle 31 and the outer tube 33 is information with which positions with respect to the probe 22, which obtains a cross-sectional image, can be identified.

Next, the control unit 60 calculates three-dimensional coordinates of the puncture position (step S2). The puncture position includes at least one of a puncture skin position S, which is a position on the skin to be punctured, a front wall puncture planned position P1, which is a position on the front wall FW to be punctured (a position where a puncture route intersects the front wall FW of the blood vessel), a rear wall puncture planned position P2, which is a position on the rear wall BW to be punctured (a position where the puncture route intersects the rear wall BW of the blood vessel), a center of gravity G of the blood vessel through which the needle 31 passes, a puncture completion planned position A1, and a radius B. The puncture position is a position planned before the puncture, and may be different from a position at which the puncture is actually performed. Note that the puncture position can be a position at which the puncture is actually performed. The puncture completion planned position A1 is the deepest position where the needle tip 32 of the needle 31 is planned to reach. In order to identify a position of a blood vessel in an image, the control unit 60 can prepare a large number of images of the same type and use a method of machine learning or deep planning. In addition, it is also possible to detect a region with blood flow by a Doppler method in the probe 22 and recognize the region as a region of a blood vessel. The control unit 60 can also calculate the puncture route using coordinates of the center of gravity G of the blood vessel and coordinates of an initial position of the needle 31. Since the probe 22, which is an ultrasonic probe, cannot see the direction in which the blood vessel extends, it is difficult to accurately grasp the positions where the puncture route intersects the front wall FW and the rear wall BW of the blood vessel from a cross-sectional image having an imaging range at a certain position. The control unit 60, however, can predict a position of the blood vessel extending in the extending direction, and can also recognize the position of the blood vessel extending in the extending direction by actually moving the probe 22 in the extending direction while contacting the skin.

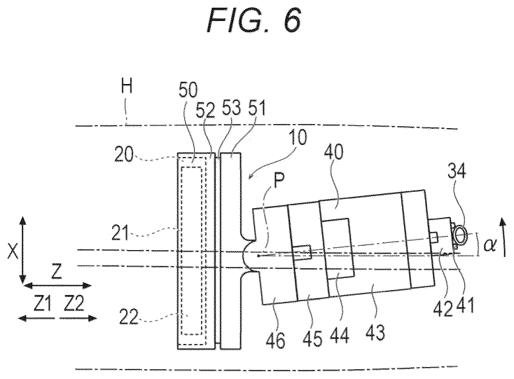

Next, as illustrated in FIGS. 5 and 6, the control unit 60 calculates a puncture speed, a puncture angle θ, and a target puncture depth L from the puncture skin position S on the skin surface and positional information regarding the blood vessel (step S3). The puncture angle θ is an angle at which the needle 31 at a time of puncture is inclined with respect to a perpendicular line of the skin surface. The puncture angle θ may be, for example, a preset angle (for example, 30 degrees), instead. The target puncture depth L is a distance from the puncture skin position S on the skin surface to the puncture completion planned position A1 after passing through the front wall puncture planned position P1, the center of gravity G of the blood vessel, and the rear wall puncture planned position P2. Note that at least one of the front wall puncture planned position P1, the center of gravity G of the blood vessel, the rear wall puncture planned position P2, and the puncture completion planned position A1 may be changed on the basis of calculation by the control unit 60 in accordance with a situation at the time of puncture.

A distance from the rear wall BW of the blood vessel to the puncture completion planned position A1 is preferably long enough and not too long for a distal end of the outer tube 33 to penetrate the rear wall BW after the needle 31 penetrates the rear wall BW.

The control unit 60 defines coordinates of the center of gravity G of the blood vessel detected by the imaging unit 20 as (x, y, 0). Next, the control unit 60 calculates a position (coordinates) and a posture (angle) of the puncture unit 30 desirable for puncture. The control unit 60 also calculates a preparation position T and a rotation angle α. The preparation position T is a position of the needle tip 32 immediately before puncture. The rotation angle α is an angle at which the needle 31 at the time of puncture is inclined with respect to the Z direction when the surface of the arm H is viewed from a perpendicular direction. The rotation angle a is set within a range in which the needle tip 32 of the needle 31 can reach the inside of an artery. The preparation position T is set at a certain height from the skin surface. The preparation position T is a position in the cross-sectional image where the needle 31 can reach the inside of the blood vessel by protruding in the extending direction (puncture direction).

Next, the control unit 60 controls and drives at least one of the first linear movement portion 42, the second linear movement portion 48, the third linear movement portion 45, the tilting portion 43, and the rotation portion 46 such that the needle 31 satisfies the puncture angle θ and the rotation angle α. As a result, the puncture unit 30 is positioned at a desired position (coordinates) with a desired posture (angle) (step S4). At this time, the needle tip 32 of the needle 31 is disposed at the preparation position T. In order to maintain a relative positional relationship between the needle 31 and the outer tube 33, the first linear movement portion 42 and the second linear movement portion 48 synchronously move the same length in the same direction. The needle 31 is arranged in such a way as to pass through the center of gravity G in the cross-sectional image.

Next, the control unit 60 controls the first linear movement portion 42 and the second linear movement portion 48 to start integral movement of the needle 31 and the outer tube 33 toward the puncture completion planned position A1 (step S5). The control unit 60 receives an instruction to start puncture from an operator using an input means such as a switch, a keyboard, or a mouse (not illustrated) connected to the control unit 60. Upon receiving the instruction, the control unit 60 drives the first linear movement portion 42 and the second linear movement portion 48.

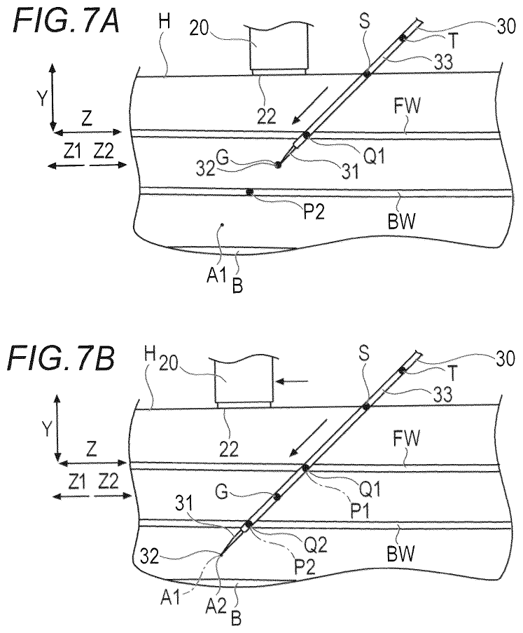

Since the control unit 60 controls the first linear movement portion 42 during a puncture operation, three-dimensional coordinates of the needle tip 32 of the needle 31 are recognized. The control unit 60 confirms that the needle tip 32 of the needle 31 passes through the center of gravity G of the blood vessel or the vicinity of the center of gravity G of the blood vessel from a latest cross-sectional image obtained from the imaging unit 20 (step S6). As illustrated in FIG. 7A, the control unit 60 continues the movement of the needle 31 and the outer tube 33 until the needle tip 32 of the needle 31 passes through the center of gravity G in the blood vessel and passes near the center of gravity G after passing through a front wall puncture position Q1. Note that the needle 31 need not strictly pass through the center of gravity G, and may pass through, for example, a position within a preset allowable range from the center of gravity G. The front wall puncture position Q1 is a position where the front wall FW is actually punctured, and may coincide with or different from the front wall puncture planned position P1. If determining that the needle tip 32 of the needle 31 has passed through the center of gravity G of the blood vessel or the vicinity of the center of gravity G of the blood vessel, the control unit 60 operates the movement unit 50 while continuing the movement of the needle 31 and the outer tube 33. As a result, as illustrated in FIG. 7B, the control unit 60 moves the imaging unit 20 from a position where the center of gravity G through which the needle 31 passes can be imaged to a position where the rear wall puncture planned position P2 can be imaged (step S7). At this time, since the imaging unit 20 is supported by the buffer 54 and is movable in the Y direction by the linear slider 27, it is possible to keep the probe 22 from being separated from the skin.

The movement of the imaging unit 20 to the position where the rear wall puncture planned position P2 can be imaged is performed before or at the same time as the needle tip 32 of the needle 31 reaches the rear wall BW of the blood vessel. The control unit 60, therefore, can determine whether or not the needle tip 32 of the needle 31 has passed through the rear wall BW by observing in real-time the cross-sectional image obtained from the imaging unit 20. The control unit 60 may identify the position of the needle tip 32 of the needle 31 or the vicinity of the needle tip 32 from set values (the puncture skin position S, the puncture speed, the puncture angle θ, the target puncture depth L, etc.) of the puncture operation and move the imaging range of the imaging unit 20 in such a way as to follow the position of the needle tip 32 or the vicinity of the needle tip 32. Alternatively, the control unit 60 may control the movement mechanism 53 of the movement unit 50 and adjust the position of the imaging range of the imaging unit 20 such that the position of the needle tip 32 or the vicinity of the needle tip 32 is always imaged in the cross-sectional image obtained from the imaging unit 20. Note that the movement of the imaging unit 20 to the position where the rear wall puncture planned position P2 can be imaged may be performed after the needle tip 32 of the needle 31 reaches the rear wall BW of the blood vessel. In this case, the control unit 60 can determine afterwards, from the cross-sectional image obtained from the imaging unit 20, whether or not the needle tip 32 of the needle 31 has passed through the rear wall BW.

The control unit 60 checks the rear wall puncture position Q2 from the latest cross-sectional image obtained from the imaging unit 20, and determines whether or not the needle tip 32 of the needle 31 has passed through the rear wall BW of the blood vessel (step S8). The rear wall puncture position Q2 is a position at which the rear wall BW is actually punctured, and may coincide with or different from the rear wall puncture planned position P2. If determining in step S8 that the needle tip 32 of the needle 31 has passed through the rear wall BW of the blood vessel, the control unit 60 determines that the puncture has been completed, and stops the driving of the first linear movement portion 42 and the second linear movement portion 48 to stop the puncture (step S9). The needle tip 32 of the needle 31 reaches the vicinity of the puncture completion position A2. As a result, the puncture with the needle 31 is normally completed. The puncture completion position A2 is a position where the needle tip 32 has finally reached, and may coincide with or be different from the puncture completion planned position A1.

After step S9, the control unit 60 may cause the display unit 70 to indicate that the puncture has been completed. In this state, the needle tip 32 of the needle 31 and the distal end of the outer tube 33 penetrate the rear wall BW of the blood vessel. After step S9, the control unit 60 may drive the first linear movement portion 42 with the second linear movement portion 48 stopped, retract the needle 31 in a direction opposite the puncture direction while leaving the outer tube 33, and pull out the needle 31 from the outer tube 33. Since the distal end of the outer tube 33 penetrates the rear wall BW, it is possible to suppress occurrence of backflow through a lumen of the outer tube 33 even when the needle 31 is pulled out. Note that an operation for removing the needle 31 from the outer tube 33 is not automatically performed under the control of the control unit 60, but may be manually performed by the operator.

If determining in step S8 that the needle 31 has not passed through the rear wall BW of the blood vessel, the control unit 60 determines whether or not the needle 31 is outside the blood vessel in the cross-sectional image obtained from the imaging unit 20 (step S10). If determining that the needle 31 is outside the blood vessel, the control unit 60 determines that the rear wall puncture position Q2 at which the needle 31 has actually punctured the rear wall BW is deviated from the rear wall puncture planned position P2 and there is a possibility that the needle 31 has already penetrated the rear wall BW, and moves the imaging unit 20 in a proximal end direction Z2 (a direction parallel to the Z direction and opposite the puncture direction) in which the needle 31 is considered to have punctured the rear wall BW (step S11). The control unit 60 attempts to identify the rear wall puncture position Q2 at which the needle 31 has penetrated the rear wall BW from the cross-sectional image obtained from the imaging unit 20, and determines whether or not the needle 31 has passed through the rear wall BW (step S12).

If determining in step S10 that the needle 31 is not outside the blood vessel (inside the blood vessel), the control unit 60 moves the imaging unit 20 to a position where the needle tip 32 of the needle 31 can be imaged (step S13). That is, the control unit 60 moves the imaging unit 20 in a distal end direction Z1 (a direction parallel to the Z direction and toward the puncture direction), which is a direction in which the needle tip 32 of the needle 31 is located. Next, the control unit 60 determines, from the cross-sectional image obtained from the imaging unit 20, whether or not the needle 31 has penetrated the rear wall BW (step S12).

If determining in step S12 that the needle 31 has passed through the rear wall BW, the control unit 60 stops the driving of the first linear movement portion 42 and the second linear movement portion 48 to stop the puncture operation (step S9). As a result, the puncture with the needle 31 is normally completed.

If determining in step S12 that the needle 31 has not passed through the rear wall BW, the control unit 60 stops the driving of the first linear movement portion 42 and the second linear movement portion 48 to stop the puncture operation (step S14). Furthermore, the control unit 60 causes the display unit 70 to display a warning indicating that an abnormality has occurred in manual work for puncture (step S15). As a result, the control unit 60 completes the manual work without completing desired puncture.

Steps S10 to S15 can be defined as a subroutine Sub1 for outputting b1 and b2 with respect to input of a1. The subroutine Sub1 is a process for the control unit 60 to determine whether or not the needle 31 has passed through the rear wall BW when it is difficult to determine that the needle 31 has passed through the rear wall BW in the image obtained from the imaging unit 20 arranged at the position where the rear wall puncture planned position P2 can be imaged. Note that the subroutine Sub1 (steps S10 to S15) need not be provided. In this case, if determining in step S8 that the needle 31 has not passed through the rear wall BW, for example, the control unit 60 may stop the puncture operation (step S14), display, on the display unit 70, a warning indicating that the puncture has been stopped (step S15), and complete the manual work without completing the desired puncture.

As described above, the vascular puncture system 10 according to the first embodiment is a vascular puncture system 10 that punctures a blood vessel with the needle 31 using the imaging unit 20 that comes into contact with a skin surface and that obtains a cross-sectional image of a human body and the drive unit 40 to which the needle 31 having the sharp needle tip 32 is connectable and that moves the needle 31 in the puncture direction, the vascular puncture system 10 including the movement unit 50 that moves the imaging unit 20, and the control unit 60 that receives information regarding the position of the needle 31 and/or information regarding the cross-sectional image and that controls the operation of the movement unit 50, in which the control unit 60 calculates a puncture position on the blood vessel from the information regarding the position of the needle 31 and/or the information regarding the cross-sectional image, and controls the operation of the movement unit 50 in such a way as to move an imaging range of the imaging unit 20 to the calculated puncture position. As a result, since the vascular puncture system 10 can change the imaging range of the imaging unit 20, it is possible to recognize, using the cross-sectional image, a puncture state of the blood vessel at a desired position in a direction in which the blood vessel extends. Note that the information regarding the cross-sectional image is electronic information for forming the cross-sectional image or electronic information used to form the cross-sectional image, and can be transmitted and received as an electronic signal.

The control unit 60 is capable of obtaining positional information regarding the rear wall BW of the blood vessel to be punctured and moving the imaging range of the imaging unit 20 from another position in such a way as to be able to image the puncture planned position of the rear wall BW. As a result, since the vascular puncture system 10 can recognize whether or not the needle 31 has reached the rear wall BW of the blood vessel and punctured the rear wall BW, it is possible to reliably puncture the rear wall BW with the needle 31 and stop the needle 31 at a desired position.

The control unit 60 is capable of obtaining positional information regarding the center of gravity G of the blood vessel to be punctured and moving the imaging range of the imaging unit 20 from another position in such a way as to be able to image a position at or around the center of gravity G through which the needle 31 is planned to pass. As a result, since the vascular puncture system 10 can recognize whether or not the needle 31 has reached the center of gravity G of the blood vessel, it is possible to reliably puncture the blood vessel with the needle 31 such that the needle passes through the blood vessel and stop the needle 31 at a desired position.

The control unit 60 controls the movement unit 50 in such a way as to move the imaging range of the imaging unit 20 before the puncture operation for controlling the drive unit 40 in such a way as to move the needle 31 in the puncture direction in order to puncture the blood vessel with the needle 31. As a result, since the vascular puncture system 10 can set the imaging range in advance at a position to be imaged, it is possible to reliably grasp a state of the blood vessel while observing changes in the state of the blood vessel to be imaged.

The control unit 60 controls the movement unit 50 in such a way as to move the imaging range of the imaging unit 20 during the puncture operation for controlling the drive unit 40 in such a way as to move the needle 31 in the puncture direction in order to puncture the blood vessel with the needle 31. As a result, the vascular puncture system 10 can reduce time required for the movement of the imaging range and the puncture operation to reduce a burden on the patient. Furthermore, since the vascular puncture system 10 can move the imaging range in accordance with the puncture operation, it is possible to continue to image a predetermined position of the needle 31, which moves in the puncture operation, and reliably grasp the state of the blood vessel in real-time.

The control unit 60 may control the operation of the movement unit 50 in accordance with a setting value for driving the drive unit 40. As a result, the vascular puncture system 10 can move the imaging range in accordance with the movement of the needle 31 driven by the drive unit 40.

The control unit 60 may control the operation of the movement unit 50 such that the position of the needle 31 identified from the cross-sectional image obtained from the imaging unit 20 is maintained at a predetermined position. As a result, the vascular puncture system 10 can move the imaging range in accordance with the movement of the needle 31 driven by the drive unit 40.

The movement unit 50 moves the imaging unit 20 in such a way as to translate the cross-sectional image. As a result, the vascular puncture system 10 can observe an entire imaging range under uniform conditions, it is possible to satisfactorily grasp a state of an observation target.

The movement unit 50 includes a buffer that supports the imaging unit 20 while absorbing displacement of the imaging unit 20 in a direction toward an imaging target. As a result, when the imaging range is changed, the vascular puncture system 10 can keep the imaging unit 20 from being separated from the imaging target and becoming unable to obtain the image.

The control unit 60 causes, if the imaging by the imaging unit 20 is abnormal, the display unit 70 (information transmission unit) that transmits information to an outside to transmit information indicating a warning. As a result, the vascular puncture system 10 can transmit a warning to the operator, and safety can be improved. Note that the information transmission unit is not limited to the display unit 70 capable of displaying an image, and may be, for example, a speaker or the like that emits sound, instead.

Furthermore, a method for controlling the vascular puncture system 10 according to the present embodiment is a method for controlling the vascular puncture system 10 that includes the imaging unit 20 which comes into contact with the skin surface and which obtains the cross-sectional image of the human body, the drive unit 40 to which the needle 31 having the sharp needle tip 32 is connectable and which moves the needle 31 in the puncture direction, the movement unit 50 which moves the imaging unit 20, and the control unit 60 which receives information regarding the position of the needle 31 and/or information regarding the cross-sectional image and which controls the operation of the drive unit 40 and the movement unit 50 and that punctures a blood vessel with the needle 31, the method being performed by the control unit 60 and including the steps of calculating the puncture position on the blood vessel from the information regarding the position of the needle 31 and/or the information regarding the cross-sectional image, and controlling the operation of the movement unit 50 in such a way as to move the imaging range of the imaging unit 20 to the calculated puncture position. As a result, since the method for controlling the vascular puncture system 10 changes the imaging range of the imaging unit 20, it is possible to recognize, using the cross-sectional image, the puncture state of the blood vessel at a desired position in the direction in which the blood vessel extends.

Furthermore, the vascular puncture system 10 according to the present embodiment is the vascular puncture system 10 that punctures the blood vessel with the needle 31 having the sharp needle tip 32, the vascular puncture system 10 including the imaging unit 20 that comes into contact with the skin surface and that obtains the cross-sectional image of the human body, the drive unit 40 to which the needle 31 is connectable and that moves the needle 31 to the puncture position on the blood vessel, the movement unit 50 that moves the imaging unit 20, and the control unit 60 that receives information regarding the position of the needle 31 and/or information regarding the cross-sectional image and that controls operation of the drive unit 40 and the movement unit 50, in which the control unit 60 calculates the puncture position on the blood vessel from the information regarding the position of the needle 31 and/or the information regarding the cross-sectional image, and controls the operation of the movement unit 50 in such a way as to move the imaging range of the imaging unit 20 to the calculated puncture position. As a result, since the vascular puncture system 10 can change the imaging range of the imaging unit 20, it is possible to recognize, using the cross-sectional image, a puncture state of the blood vessel at a desired position in a direction in which the blood vessel extends.

### Second Embodiment

A vascular puncture system 10 according to a second embodiment is different from that according to the first embodiment in terms of content of the control performed the control unit 60. In the second embodiment, unlike in the first embodiment in which the control unit 60 moves the imaging unit 20 to the position where the rear wall puncture planned position P2 can be imaged after starting the puncture operation with the needle 31 using the drive unit 40, the control unit 60 moves the imaging unit 20 to the position where the rear wall puncture planned position P2 can be imaged before starting the puncture operation with the needle 31 using the drive unit 40.

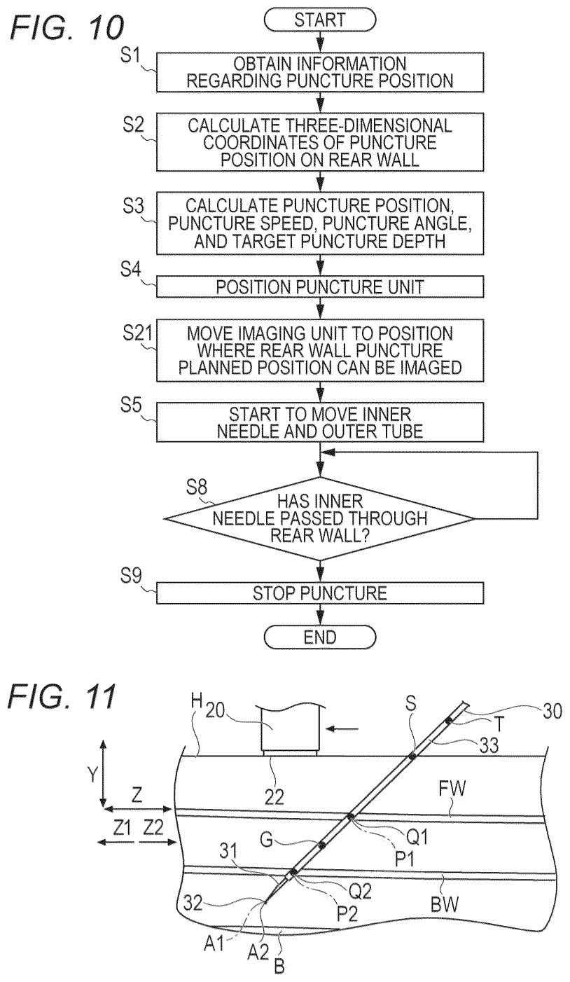

Next, a method for puncturing a blood vessel using the vascular puncture system 10 according to the second embodiment will be described with reference to a flowchart of FIG. 10 used by the control unit 60. Note that steps similar to those in the first embodiment are given the same reference numerals, and description of those steps similar to those in first embodiment are omitted or simplified.

The control unit 60 obtains information regarding a puncture position determined by another program (step S1). Next, the control unit 60 calculates three-dimensional coordinates of the puncture skin position S, the front wall puncture planned position P1, the rear wall puncture planned position P2, the center of gravity G of a blood vessel through which the needle 31 passes, the puncture completion planned position A1, the radius B, and the like (step S2). Next, as illustrated in FIGS. 5 and 6, the control unit 60 calculates a puncture speed, a puncture angle 0, and a target puncture depth L from the puncture skin position S on the skin surface and positional information regarding the blood vessel (step S3).

Next, the control unit 60 controls at least one of the first linear movement portion 42, the second linear movement portion 48, the third linear movement portion 45, the tilting portion 43, and the rotation portion 46 to position the puncture unit 30 at a desired position (coordinates) in a desired posture (angle) (step S4).

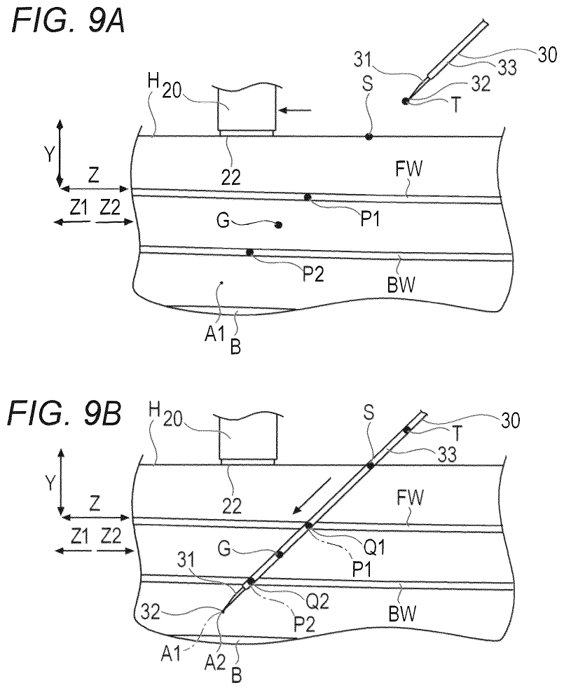

Next, as illustrated in FIG. 9A, the control unit 60 moves the imaging unit 20 to the position where the rear wall puncture planned position P2 of the blood vessel can be imaged (step S21). Next, the control unit 60 controls the first linear movement portion 42 and the second linear movement portion 48 to start integral movement of the needle 31 and the outer tube 33 toward the puncture completion planned position A1 (step S5).

The control unit 60 continues the movement of the needle 31 and the outer tube 33, and determines, from the cross-sectional image obtained from the imaging unit 20, whether or not the needle tip 32 of the needle 31 has passed through the rear wall BW (step S8). If determining that the needle tip 32 of the needle 31 has passed through the rear wall BW of the blood vessel, the control unit 60 determines that the puncture has been completed, and stops the driving of the first linear movement portion 42 and the second linear movement portion 48 to stop the puncture (step S9). As a result, the puncture with the needle 31 is normally completed.

### Third Embodiment

A vascular puncture system 10 according to a third embodiment is different

from that according to the first embodiment in terms of the content of the control performed the control unit 60. In the third embodiment, unlike in the first embodiment in which the control unit 60 moves the imaging unit 20 to the position where the rear wall puncture planned position P2 can be imaged after starting the puncture operation with the needle 31 using the drive unit 40, the control unit 60 moves the imaging unit 20 to a position where the rear wall puncture position Q2 can be imaged after completing the puncture operation with the needle 31 using the drive unit 40.

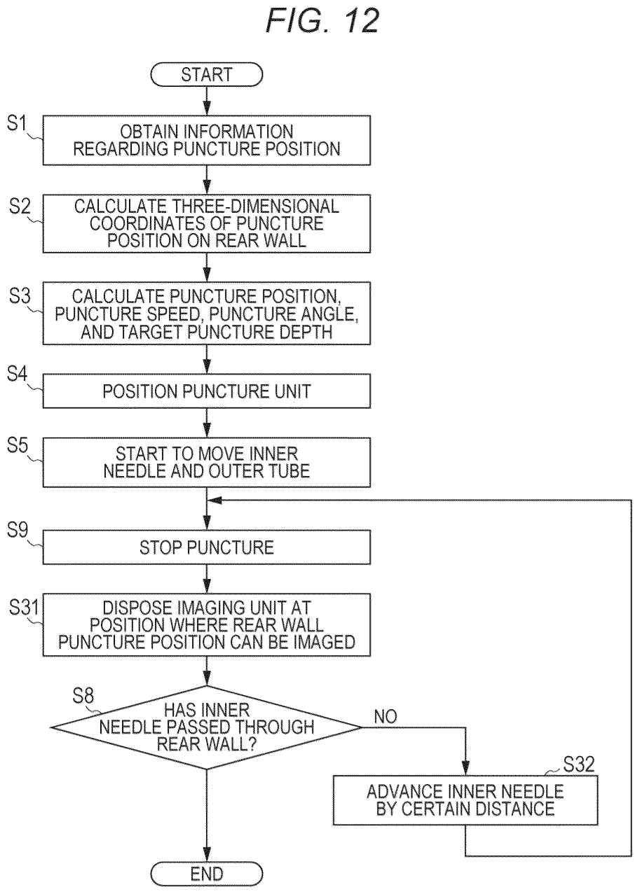

Next, a method for puncturing a blood vessel using the vascular puncture system 10 according to the third embodiment will be described with reference to a flowchart of FIG. 12 used by the control unit 60. Note that steps similar to those in the first embodiment are given the same reference numerals, and description steps similar to those in the first embodiment are omitted or simplified.

The control unit 60 obtains information regarding a puncture position determined by another program (step S1). Next, the control unit 60 calculates three-dimensional coordinates of the puncture skin position S, the front wall puncture planned position P1, the rear wall puncture planned position P2, the center of gravity G of a blood vessel through which the needle 31 passes, the puncture completion planned position A1, the radius B, and the like (step S2). Next, as illustrated in FIGS. 5 and 6, the control unit 60 calculates a puncture speed, a puncture angle θ, and a target puncture depth L from the puncture skin position S on the skin surface and positional information regarding the blood vessel (step S3).

Next, the control unit 60 controls at least one of the first linear movement portion 42, the second linear movement portion 48, the third linear movement portion 45, the tilting portion 43, and the rotation portion 46 to position the puncture unit 30 at a desired position (coordinates) in a desired posture (angle) (step S4).

Next, the control unit 60 controls the first linear movement portion 42 and the second linear movement portion 48 to start integral movement of the needle 31 and the outer tube 33 toward the puncture completion planned position A1 (step S5). Next, in a state where the needle tip 32 of the needle 31 has reached the puncture completion position A2, the control unit 60 stops the driving of the first linear movement portion 42 and the second linear movement portion 48 to stop the puncture (step S9).

As illustrated in FIG. 11, the control unit 60 controls the movement unit 50 to dispose the imaging unit 20 at the position where the rear wall puncture planned position P2 can be imaged (step S31). Note that the control unit 60 may dispose the imaging unit 20 at the position where the rear wall puncture position Q2 at which the puncture has been actually performed can be imaged while moving the imaging unit 20 using the movement unit 50 and checking the cross-sectional image. Next, the control unit 60 determines, from the cross-sectional image obtained from the imaging unit 20, whether or not the needle tip 32 of the needle 31 has passed through the rear wall BW (step S8). If determining in step S8 that the needle tip 32 of the needle 31 has passed through the rear wall BW of the blood vessel, the control unit 60 determines that the puncture has been completed, and stops the driving of the first linear movement portion 42 and the second linear movement portion 48 to stop the puncture (step S9). As a result, the puncture with the needle 31 is normally completed.

If determining in step S8 that the needle 31 has not passed through the rear wall BW of the blood vessel, the control unit 60 controls the first linear movement portion 42 and the second linear movement portion 48 in such a way as to advance the needle 31 and the outer tube 33 in the puncture direction by a certain distance (step S32). Next, the control unit 60 stops the driving of the first linear movement portion 42 and the second linear movement portion 48 to stop the puncture operation (step S9), disposes the imaging unit 20 at the position where the rear wall puncture planned position P2 or the rear wall puncture position Q2 can be imaged (step S31), and determines whether or not the needle 31 is outside the blood vessel (step S10). If the control unit 60 determines in step S8 that the needle tip 32 of the needle 31 has passed through the rear wall BW of the blood vessel, the puncture with the needle 31 is normally completed while assuming that the puncture has been normally performed. The control unit 60 then repeats the above-described steps S32, S9, S31, and S8 until determining in step S8 that the needle 31 has passed through the rear wall BW of the blood vessel. As a result, the puncture with the needle 31 is completed.

As described above, in the third embodiment, the control unit 60 controls the movement unit 50 in such a way as to move the imaging range of the imaging unit 20 after the puncture operation for controlling the drive unit 40 in such a way as to move the needle 31 in the puncture direction in order to puncture the blood vessel with the needle 31. As a result, since the vascular puncture system 10 can set the imaging range in advance at the position to be imaged, it is possible to reliably grasp a state of the position to be imaged.

### Fourth Embodiment

A vascular puncture system 10 according to a fourth embodiment is different from that according to the first embodiment in terms of the content of the control performed the control unit 60. In the fourth embodiment, the control unit 60 moves the imaging unit 20 to a position where the front wall puncture planned position P1 can be imaged, a position at or around the center of gravity G through which the needle 31 is planned to pass, and the position where the rear wall puncture planned position P2 can be imaged, and determines passage of the needle 31 at each position.

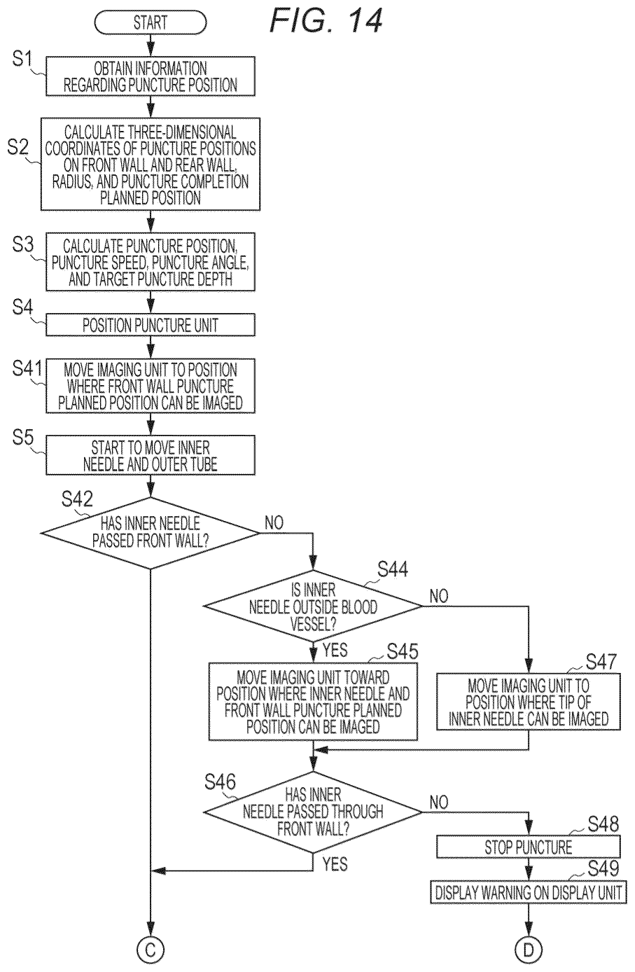

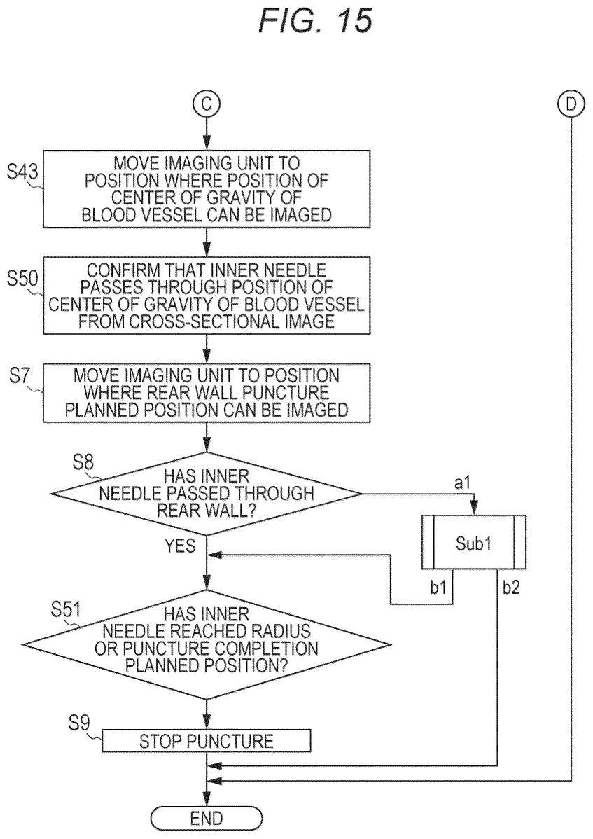

Next, a method for puncturing a blood vessel using the vascular puncture system 10 according to the fourth embodiment will be described with reference to flowcharts of FIGS. 14 and 15 used by the control unit 60. Note that steps similar to those in the first embodiment are given the same reference numerals, and description of steps similar to those in the first embodiment are omitted or simplified.

The control unit 60 obtains information regarding a puncture position determined by another program (step S1). Next, the control unit 60 calculates three-dimensional coordinates of the puncture skin position S, the front wall puncture planned position P1, the rear wall puncture planned position P2, the center of gravity G of a blood vessel through which the needle 31 passes, the puncture completion planned position A1, the radius B, and the like (step S2). Next, as illustrated in FIGS. 5 and 6, the control unit 60 calculates a puncture speed, a puncture angle θ, and a target puncture depth L from the puncture skin position S on the skin surface and positional information regarding the blood vessel (step S3).

Next, the control unit 60 controls at least one of the first linear movement portion 42, the second linear movement portion 48, the third linear movement portion 45, the tilting portion 43, and the rotation portion 46 to position the puncture unit 30 at a desired position (coordinates) in a desired posture (angle) (step S4).

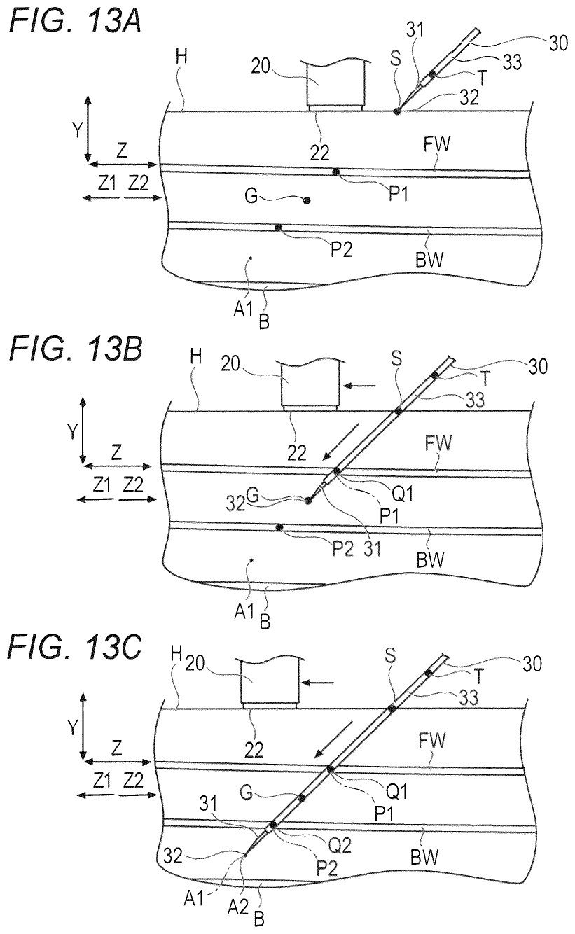

Next, as illustrated in FIG. 13A, the control unit 60 moves the imaging unit 20 to the position where the image of the front wall puncture planned position P1 can be captured (step S41). Next, the control unit 60 controls the first linear movement portion 42 and the second linear movement portion 48 to start integral movement of the needle 31 and the outer tube 33 toward the puncture completion planned position A1 (step S5).

The control unit 60 determines, from the cross-sectional image obtained from the imaging unit 20, whether or not the needle tip 32 of the needle 31 has passed through the front wall FW (step S42).

If determining in step S42 that the needle tip 32 of the needle 31 has passed through the front wall FW of the blood vessel, the control unit 60 determines that the puncture of the front wall FW is completed, and moves the imaging unit 20 to the position at or around the center of gravity G of the blood vessel through which the needle 31 passes can be imaged as illustrated in FIG. 13B (step S43).

If determining in step S42 that the needle 31 has not passed through the front wall FW of the blood vessel, the control unit 60 determines whether or not the needle 31 is outside the blood vessel in the cross-sectional image obtained from the imaging unit 20 (step S44). If determining that the needle 31 is outside the blood vessel, the control unit 60 determines that the front wall puncture position Q1 where the needle 31 has actually punctured the front wall FW is deviated from the front wall puncture planned position P1 and there is a possibility that the needle 31 has already penetrated the front wall FW, and moves the imaging unit 20 in the distal end direction Z1 (or the proximal end direction Z2) in which the needle 31 is considered to have punctured the front wall FW (step S45). The control unit 60 attempts to identify the front wall puncture position Q1 at which the needle 31 has penetrated the front wall FW from the cross-sectional image obtained from the imaging unit 20, and determines whether or not the needle 31 has passed through the front wall FW (step S46).

If determining in step S44 that the needle 31 is not outside the blood vessel (inside the blood vessel), the control unit 60 moves the imaging unit 20 to the position where the needle tip 32 of the needle 31 can be imaged (step S47). That is, the control unit 60 moves the imaging unit 20 in the distal end direction Z1, which is the direction in which the needle tip 32 of the needle 31 is located. Next, the control unit 60 determines, from the cross-sectional image obtained from the imaging unit 20, whether or not the needle 31 has penetrated the front wall FW (step S46).

If determining in step S46 that the needle 31 has passed through the front wall FW, the control unit 60 determines that the puncture of the front wall FW has been completed, and moves the imaging unit 20 to the position at or around the center of gravity G of the blood vessel through which the needle 31 passes can be imaged (step S43).

If determining in step S46 that the needle 31 has not passed through the front wall FW, the control unit 60 stops the driving of the first linear movement portion 42 and the second linear movement portion 48 to stop the puncture operation (step S48). Furthermore, the control unit 60 causes the display unit 70 to display a warning indicating that an abnormality has occurred in manual work for puncture (step S49). As a result, the control unit 60 completes the manual work without completing desired puncture.

After moving, in step S43, the imaging unit 20 to the position at or around the center of gravity G of the blood vessel through which the needle 31 passes can be imaged, the control unit 60 confirms that the needle 31 passes through the center of gravity G or the vicinity thereof from the cross-sectional image obtained by the imaging unit 20 (step S50). Next, as illustrated in FIG. 13C, the control unit 60 moves the imaging unit 20 to the position where the rear wall puncture planned position P2 can be imaged (step S7). Next, the control unit 60 determines, from the cross-sectional image obtained from the imaging unit 20, whether or not the needle tip 32 of the needle 31 has passed through the rear wall BW (step S8).

If determining in step S8 that the needle tip 32 of the needle 31 has passed through the rear wall BW of the blood vessel, the control unit 60 moves the needle 31 and the outer tube 33 until the needle tip 32 of the needle 31 reaches the radius B or the puncture completion position A2. If determining that the needle tip 32 of the needle 31 has reached the radius B or the puncture completion position A2 (step S51), the control unit 60 determines that the puncture has been completed, and stops the driving of the first linear movement portion 42 and the second linear movement portion 48 to stop the puncture (step S9). As a result, the puncture with the needle 31 is normally completed.

If determining in step S8 that the needle 31 has not passed through the rear wall BW of the blood vessel, the control unit 60 can perform calculation of the subroutine Sub1 as in the first embodiment. The subroutine Sub1 is a process for the control unit 60 to determine whether or not the needle 31 has passed through the rear wall BW when it is difficult to determine that the needle 31 has passed through the rear wall BW in the image obtained from the imaging unit 20 arranged at the position where the rear wall puncture planned position P2 can be imaged.

As described above, in the fourth embodiment, the control unit 60 can obtain positional information regarding the front wall FW of the blood vessel to be punctured and move the imaging range of the imaging unit 20 from another position such that the puncture planned position P1 of the front wall FW can be imaged. As a result, the vascular puncture system 10 can recognize whether or not the needle 31 has reached the front wall FW of the blood vessel and punctured the front wall FW, so that the front wall FW can be reliably punctured with the needle 31, and the needle 31 can be stopped at a desired position.

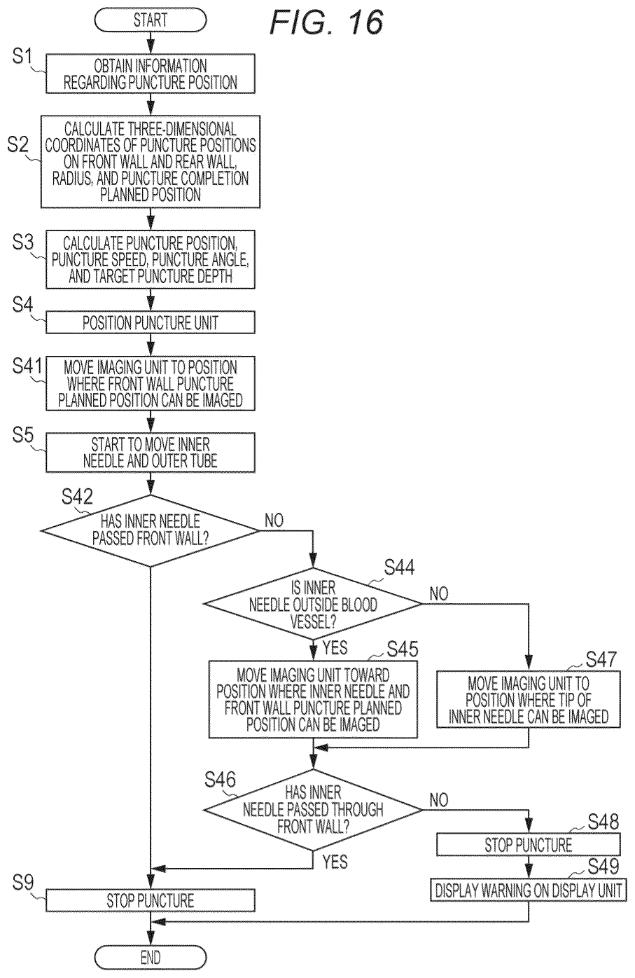

Note that the present disclosure is not limited to the embodiments described above, and those skilled in the art can make various modifications within the technical idea of the present disclosure. For example, as illustrated in FIG. 16, the control unit 60 may perform only the control flow corresponding to the puncture operation for the front wall FW among the control flows according to the fourth embodiment. The vascular puncture system 10, therefore, may be used for so-called single-wall puncture (SWP), in which the needle 31 punctures only the front wall FW of the blood vessel.

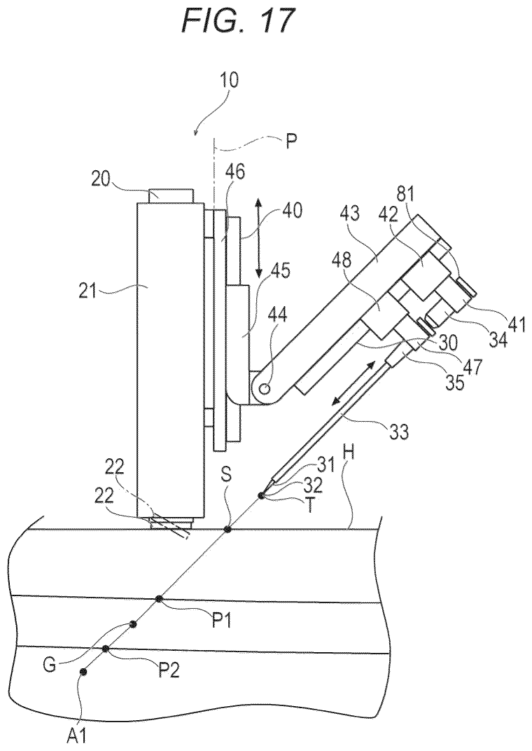

In addition, as in a modification illustrated in FIG. 17, the movement of the imaging range of the imaging unit 20 may be performed by tilting a portion including the probe 22. In this case, the movement unit 50 includes a drive source that tilts the portion including the probe 22. The movement unit 50, therefore, may move the imaging unit 20 such that the cross-sectional image is tilted. As a result, the vascular puncture system 10 need not slide the imaging unit 20 with respect to the imaging target. Consequently, the imaging range can be smoothly changed, and when the imaging range is changed, it is possible to keep the imaging unit 20 from being separated from the arm H to be imaged and becoming unable to obtain the image.

In addition, the movement unit 50 may include a drive source for tilting the probe 22 while translating the imaging range of the imaging unit 20 in the Z direction, which is the length direction of the arm H. In addition, the movement unit 50 may include a drive source for translating the imaging range of the imaging unit 20 in the Y direction.

The detailed description above describes embodiments of a vascular puncture system capable of detecting a position of a blood vessel from an image obtained by an echograph and puncturing the blood vessel, and a method for controlling the vascular puncture system. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A vascular puncture system that punctures a blood vessel with a needle using an imaging unit configured to come into contact with a skin surface and to obtain a cross-sectional image of a human body and a drive unit to which a needle having a sharp needle tip is connectable and which moves the needle in a puncture direction, the vascular puncture system comprising:
a movement unit configured to move the imaging unit; and
a control unit configured to:
receive information regarding a position of the needle and/or information regarding the cross-sectional image and that controls operation of the movement unit;
calculate a puncture position on the blood vessel from the information regarding the position of the needle and/or the information regarding the cross-sectional image; and
control the operation of the movement unit in such a way as to move an imaging range of the imaging unit to the calculated puncture position.

2. The vascular puncture system according to claim 1, wherein the control unit is configured to obtain positional information regarding a rear wall of the blood vessel to be punctured and to move the imaging range of the imaging unit from another position in such a way as to be able to image a puncture planned position of the rear wall.

3. The vascular puncture system according to claim 1, wherein the control unit is configured to obtain positional information regarding a center of gravity of the blood vessel to be punctured and to move the imaging range of the imaging unit from another position in such a way as to be able to image a position at or around the center of gravity through which the needle is planned to pass.

4. The vascular puncture system according to claim 1, wherein the control unit is configured to obtain positional information regarding a front wall of the blood vessel to be punctured and to move the imaging range of the imaging unit from another position in such a way as to be able to image a puncture planned position of the front wall.

5. The vascular puncture system according to claim 1, wherein the control unit is configured to control the movement unit in such a way as to move the imaging range of the imaging unit before a puncture operation for controlling the drive unit in such a way as to move the needle in the puncture direction in order to puncture the blood vessel with the needle.

6. The vascular puncture system according to claim 1, wherein the control unit is configured to control the movement unit in such a way as to move the imaging range of the imaging unit during a puncture operation for controlling the drive unit in such a way as to move the needle in the puncture direction in order to puncture the blood vessel with the needle.

7. The vascular puncture system according to claim 1, wherein the control unit is configured to control the movement unit in such a way as to move the imaging range of the imaging unit after a puncture operation for controlling the drive unit in such a way as to move the needle in the puncture direction in order to puncture the blood vessel with the needle.

8. The vascular puncture system according to claim 6, wherein the control unit is configured to control the operation of the movement unit in accordance with a setting value for driving the drive unit.

9. The vascular puncture system according to claim 6, wherein the control unit is configured to control the operation of the movement unit such that the position of the needle identified from the cross-sectional image obtained from the imaging unit is maintained at a predetermined position.

10. The vascular puncture system according to claim 1, wherein the movement unit is configured to move the imaging unit in such a way as to translate the cross-sectional image.

11. The vascular puncture system according to claim 1, wherein the movement unit is configured to move the imaging unit in such a way as to tilt the cross-sectional image.

12. The vascular puncture system according to claim 1, wherein the movement unit includes a buffer, the buffer configured to support the imaging unit while absorbing displacement of the imaging unit in a direction toward an imaging target.

13. The vascular puncture system according to claim 1, wherein the control unit is configured to cause, if the imaging by the imaging unit is abnormal, an information transmission unit that transmits information to an outside to transmit information indicating a warning.

14. A method for controlling a vascular puncture system that includes an imaging unit which comes into contact with a skin surface and which obtains a cross-sectional image of a human body, a drive unit to which a needle having a sharp needle tip is connectable and which moves the needle in a puncture direction, a movement unit which moves the imaging unit, and a control unit which receives information regarding a position of the needle and/or information regarding the cross-sectional image and which controls operation of the drive unit and the movement unit and that punctures a blood vessel with the needle, the method being performed by the control unit and comprising:
calculating a puncture position on the blood vessel from the information regarding the position of the needle and/or the information regarding the cross-sectional image; and
controlling the operation of the movement unit in such a way as to move an imaging range of the imaging unit to the calculated puncture position.

15. The method according to claim 14, further comprising:
obtaining, by the control unit, positional information regarding a rear wall of the blood vessel to be punctured and to move the imaging range of the imaging unit from another position in such a way as to be able to image a puncture planned position of the rear wall.

16. The method according to claim 14, further comprising:
obtaining, by the control unit, positional information regarding a center of gravity of the blood vessel to be punctured and to move the imaging range of the imaging unit from another position in such a way as to be able to image a position at or around the center of gravity through which the needle is planned to pass.

17. The method according to claim 14, further comprising:
obtaining, by the control unit, positional information regarding a front wall of the blood vessel to be punctured and to move the imaging range of the imaging unit from another position in such a way as to be able to image a puncture planned position of the front wall.

18. The method according to claim 14, further comprising:
controlling, by the control unit, the movement unit in such a way as to move the imaging range of the imaging unit before a puncture operation for controlling the drive unit in such a way as to move the needle in the puncture direction in order to puncture the blood vessel with the needle;
controlling, by the control unit, the movement unit in such a way as to move the imaging range of the imaging unit during a puncture operation for controlling the drive unit in such a way as to move the needle in the puncture direction in order to puncture the blood vessel with the needle; and
controlling, by the control unit, the movement unit in such a way as to move the imaging range of the imaging unit after a puncture operation for controlling the drive unit in such a way as to move the needle in the puncture direction in order to puncture the blood vessel with the needle.

19. The method according to claim 18, further comprising:
controlling, by the control unit, the operation of the movement unit in accordance with a setting value for driving the drive unit.

20. A vascular puncture system configured to puncture a blood vessel with a needle having a sharp needle tip, the vascular puncture system comprising:
an imaging unit configured to come into contact with a skin surface and to obtain a cross-sectional image of a human body;
a drive unit to which the needle is connectable and configured to move the needle to a puncture position on the blood vessel;
a movement unit configured to move the imaging unit; and
a control unit configured to:
receive information regarding a position of the needle and/or information regarding the cross-sectional image and that controls operation of the drive unit and the movement unit;
calculate the puncture position on the blood vessel from the information regarding the position of the needle and/or the information regarding the cross-sectional image; and
control the operation of the movement unit in such a way as to move an imaging range of the imaging unit to the calculated puncture position.
